(19) United States
(12) Patent Application Publication
(10) Pub. No.: US 2011/0064702 A1
(43) Pub. Date: Mar. 17, 2011

(54) Methods and Kits for Enhancing Cell Survival, Stimulating Cell Proliferation, Treating Diabetic Patients, and/or Reinnervation

(76) Inventors: Elena Kozlova, Stockholm (SE); Leif Jansson, Sala (SE)

(21) Appl. No.: 12/881,407

(22) Filed: Sep. 14, 2010

Related U.S. Application Data
(60) Provisional application No. 61/242,190, filed on Sep. 14, 2009.

(51) Int. Cl. A61K 35/12; A61P 3/10

(52) U.S. Cl. 424/93.7

(57) ABSTRACT

Methods for enhancing beta cell survival and/or for stimulating beta cell proliferation, comprise co-transplanting pancreatic islets, beta cells, and/or stem cells which can generate beta cells, with (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells. Methods for reinnervation in an organ or tissue transplant patient comprise co-transplanting with the organ or tissue (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells. Kits for conducting such methods employ at least one of (i) neural crest stem cells (NCSCs), (ii) tetracycline-regulated gene expression system (Tet-System)-containing neural stem/progenitor cells (TetStock neural stem/progenitor cells), and (iii) pre-differentiated stem/progenitor neural cells.

### RELATED APPLICATIONS

The present application claims priority under 35 U.S.C. §119 of U.S. Application Ser. No. 61/242,190 filed Sep. 14, 2009.

### FIELD OF THE INVENTION

The present invention is directed to methods for enhancing cell survival and/or stimulating cell proliferation, particularly beta cell survival and/or proliferation, for example during and/or after transplantation. In a specific embodiment, the invention is directed to methods for treating type 1 diabetic or type 2 insulin-deficient diabetic patients. The present invention is also directed to methods and kits for reinnervation in an organ or tissue transplant patient. The methods and kits employ (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells.

### BACKGROUND OF THE INVENTION

Transplantation of either the whole pancreas or isolated islets of Langerhans (pancreatic islets) has become a treatment of choice for selected patients with diabetes mellitus (Frank et al, “Comparison of whole organ pancreas and isolated islet transplantation for type 1 diabetes,” Adv Surg, 39:137-163 (2005); Ryan et al, “Current indications for pancreas or islet transplant,” Diabetes Obes Metab, 8:1-7 (2006)).

Long-term results after islet transplantation are disappointing, with adequate graft function seen in less than 10% of patients after five years (Ryan et al, “Five-year follow-up after clinical islet transplantation,” Diabetes, 54:2060-2069 (2005)), even though the one-year survival has been almost 90% (Shapiro et al, “Islet transplantation in seven patients with type 1 diabetes mellitus using a glucocorticoid-free immunosuppressive regimen,” N Engl J Med, 343:230-238 (2000)). There are various reasons for graft failure, and an important contributor is the immediate post-transplantation cell death due to hypoxia, leading to a decrease in the number of engrafted beta cells (Davalli et al, “Vulnerability of islets in the immediate post transplantation period. Dynamic changes in structure and function,” Diabetes 45:1161-1167 (1996)). This, in turn, leads to the presence of only a marginal beta cell mass which becomes vulnerable to deficiencies in growth post-transplantation leading to long-term graft failure (Hellerström et al, “Experimental pancreatic transplantation in diabetes,” Diabetes Care, 11 Suppl 1:45-53 (1988); Jansson et al, “Graft vascular function after transplantation of pancreatic islets,” Diabetologia, 45:749-763 (2002)).

Several growth factors can affect beta cell replication in vitro, whereas expansion of islet endocrine cell mass in vivo is more difficult to achieve (Baggio et al, “Therapeutic approaches to preserve islet mass in type 2 diabetes,” Annu Rev Med, 57:265-281 (2006); Bouwens et al, “Regulation of pancreatic beta-cell mass,” Physiol Rev, 85:1255-1270 (2005); Vasavada et al, “Growth factors and beta cell replication,” Int J Biochem Cell Biol, 38:931-950 (2006)). Previous experiments have demonstrated that beta cell mass can be expanded during certain conditions with increased demand on function, but the exact mechanisms are as yet unclear (Bouwens et al, “Regulation of pancreatic beta-cell mass,” Physiol Rev, 85:1255-1270 (2005); Bonner-Weir, “Islet growth and development in the adult,” Journal of Molecular Endocrinology, 24:297-302 (2000)). There are also several studies suggesting that neurotrophins may affect the growth of both nerves and beta cells (Miao et al, “In vitro and in vivo improvement of islet survival following treatment with nerve growth factor,” Transplantation, 81:519-524 (2006); Teitelman et al, “Islet injury induces neurotrophin expression in pancreatic cells and reactive gliosis of peri-islet Schwann cells,” J Neurobiol, 34:304-318 (1998)).

Prior research has co-cultured islets and embryonic dorsal root ganglia (DRG) and noted an improved insulin secretion by this procedure (Kozlova et al, “In vitro interactions between insulin-producing beta cells and embryonic dorsal root ganglia,” Pancreas, 31:380-384 (2005)). Recently, neural crest stem cells (NCSCs) were shown to migrate towards pancreatic islets after transplantation under the kidney capsule and that islets induce differentiation of the NCSCs towards neurons in vitro and in vivo after transplantation (Kozlova et al, “Differentiation and migration of neural crest stem cells is stimulated by pancreatic islets,” Neuroreport, 20:833-838 (2009)).

The endocrine pancreas normally possesses a rich innervation consisting of both sympathetic, parasympathetic, sensory, peptidergic, and nitric oxide synthase-containing neurons which help to modulate endocrine secretion (Ahrén, “Autonomic regulation of islet hormone secretion—implications for health and disease,” Diabetologia, 43:393-410 (2000); Brunicardi et al, “Neural regulation of the endocrine pancreas” Int J Pancreatol, 18:177-195 (1995)). After implantation, however, most nerves degenerate and are only slowly replaced by nerves growing in from surrounding structures, mainly in association with blood vessels (Korsgren et al, “Reinnervation of transplanted pancreatic islets: a comparison between islets implanted into the kidney, spleen, or liver,” Transplant Proc, 24:1025-1026 (1992); Persson-Sjögren et al, “Peptides and other neuronal markers in transplanted pancreatic islets,” Peptides, 21:741 (2000)). Furthermore, it seems as if neurons present in islet grafts do not survive for more than up to a week after transplantation (Persson-Sjögren et al (2000)).

Since islet survival after transplantation to patients with type 1 diabetes is insufficient, new strategies to enhance transplant viability and beta cell proliferation need to be developed. The current limitation of tissue for transplantation to these patients has initiated stem cell research with the purpose to produce fully functional beta cell mass from transplanted embryonic stem (ES) cells or induced pluripotent stem (iPS) cells. ES cells, as well as other types of stem cells, are potential sources for treatment of many diseases. Studies during recent years have demonstrated that pluripotent ES cells may generate specific types of desired cells after transplantation to adult recipients, opening a new avenue in human transplantation for restorative medical purposes. For example, the recently shown successful generation of beta cells from human ES cells offers new possibilities to treat type 1 diabetes and insulin-deficient type 2 diabetes (Kroon et al, “Pancreatic endoderm derived from human embryonic stem cells generates glucose-responsive insulin-secreting cells in vivo,” Nat Biotechnol, 26:443-452 (2008)). However, further developments for successful treatments are necessary.

### SUMMARY OF THE INVENTION

Accordingly, it is an object of the invention to provide methods for improving transplantation, for example, of islets, beta cells, or the like.

In one embodiment, the invention is directed to a method for enhancing beta cell survival and/or for stimulating beta cell proliferation. The method comprises co-transplanting pancreatic islets, beta cells, and/or stem cells which can generate beta cells, with (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells.

In another, more specific embodiment, the invention is directed to a method for treating a type 1 diabetic or type 2 insulin-deficient diabetic patient, comprising co-transplanting human pancreatic islets, beta cells, and/or stem cells which can generate beta cells, with neural crest stem cells (NCSCs).

In another embodiment, the invention is directed to a kit for enhancing beta cell survival and/or for stimulating beta cell proliferation, comprising pancreatic islets, beta cells, and/or stem cells which can generate beta cells, and (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells.

According to another embodiment, the invention is directed to a method for reinnervation in an organ or tissue transplant patient, comprising co-transplanting with the organ or tissue (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells. In a related embodiment, the invention is directed to a kit for reinnervation in a stem-cell containing organ or tissue transplant patient, comprising a stem-cell containing organ or tissue, and (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells.

The methods and kits according to the invention provide enhanced cell survival and/or enhance cell proliferation, particularly, beta cell survival and/or proliferation, and/or improved reinnervation, thereby improving transplantation outcomes. These and additional objects, embodiments and advantages will be further apparent in view of the detail description.

### BRIEF DESCRIPTION OF THE DRAWINGS

The following detailed description will be more fully understood in view of the drawings, in which:

FIG. 1A shows C57BL/6-b-actin-enhanced green fluorescent protein (eGFP)-expressing boundary cap NCSCs (bNCSCs) migrating under the kidney capsule, and FIG. 1B shows eGFP-expressing bNCSCs (short bold arrows) have migrated from the lower pole of the kidney and reached the islet graft (long thin arrows) in the upper pole, scale bar=50 μm, as described in the Example.

FIG. 2, top row A, shows mixed islet-bNCSC grafts, wherein eGFP-expressing bNCSCs (short bold arrows) surround groups of islet cells (long thin arrows), but distinct boundaries between the two cell types are still maintained; and, bottom row B, shows bNCSCs predominantly differentiate to glial cells as shown by double labeling of eGFP (short bold arrows) and anti-glial fibrillary acidic protein (GFAP), as described in the Example. The arrows indicate GFAP and eGFP co-expression. Scale bar=(top row A) 200 μm, (bottom row B) 50 μm.

FIG. 3 shows eGFP-expressing bNCSCs (short bold arrows), labeled with the pan-neuronal marker anti-β-tubulin (bTUB), but these cells were not associated with islet grafts, as described in the Example. The arrows indicate bTUB and eGFP co-expression. Scale bar=50 μm.

FIGS. 4A-4H show grafts two days post-transplantation, as described in the Example. Specifically, islet-alone transplant, FIG. 4A, is in close apposition to the kidney, whereas mixed transplant, FIG. 4B, displays a cyst in the middle (sections labeled for insulin and counterstained with haematoxylin). FIGS. 4C and 4D show mixed graft in which some insulin-expressing cells are completely covered by eGFP-expressing NCSCs (FIG. 4C, grey cells), whereas in other parts of the transplant NCSCs (FIG. 4D, grey cells) are located in the immediate vicinity of the tightly packed insulin-positive cells (FIG. 4D, white cells). FIGS. 4E and 4F show Ki67 labeled endocrine cells (dark-gray) in islet-alone (FIG. 4E) and mixed (FIG. 4F) transplants. FIGS. 4G and 4H show triple labeling of islet-alone (FIG. 4G, Ki67 absent in the islet-alone transplant) and a mixed graft (FIG. 4H). Insulin-positive cells are located on the top of the graft co-expressing Ki67 (FIG. 4H; arrowhead). FIG. 4I graphically shows the fraction of Ki67 labeled cells in endocrine areas of the islet-alone and mixed grafts (n=7; p<0.001 Wilcoxon's Rank-sum test). Scale bar=300 μm (FIGS. 4A and 4B), 200 μm (FIG. 4D), 100 μm (FIG. 4C) and 20 μm (FIGS. 4E-4H).

FIGS. 5A-5H show additional data one month post-transplantation, as described in the Example. FIGS. 5A and 5B show transplants of islets alone with a high proportion of insulin-positive cells and of a mixture of islets and NCSCs, respectively, scale bar 100 μm, while FIGS. 5C-5E show mixed islet—NCSCs grafts, scale bar 50 μm. FIG. 5F shows volume of beta cell population within islets-alone transplants (Islets) or a mixed islet-NCSC transplants (Islets+NCSCs) two days and one month after transplantation. Islet grafts consisted of 150 islets and mixed grafts of 75 islets+75 neurospheres. Values are means±SEM for 6-7 experiments. *p≦0.05 for Islets+NCSCs grafts at two days compared to 28 days. FIG. 5G shows perfusion of grafts containing 150 islets (filled circles) or 75 islets+75 neurospheres (open circles) were perfused (1 ml/min) in small chambers. Values are means±SEM for 6-7 experiments. All values between 30 and 60 min are higher (P<0.05) than the values at time 30 in both groups (Student's unpaired t-test; significances not given in this figure). FIG. 5H shows intravenous glucose tolerance tests in alloxan-diabetic mice one month after grafting of 300 islets alone (filled circles), or 150 islets+150 neurospheres (open circles). Both groups of animals show normal glucose tolerance at the different time points tested.

FIGS. 6A-6C show co-culture of mouse NCSCs with mouse pancreatic islets results in extensive proliferation of beta cells after 3 days of experiment (FIGS. 6A and 6B, white dots are dividing cells, marked with Ki67). After one week in culture, the islets develop a “daisy” shape with extensive proliferation of beta cells in the core of the islet (FIG. 6B). Co-culture of mouse NCSCs with human islets induces the proliferation of islet cells, including the proliferation of beta cells in vitro (FIG. 6C, grey dots).

Further details of the drawings will be more fully apparent and understood in view of the detailed description and the Example therein.

### DETAILED DESCRIPTION

The present invention is directed to, inter alia, methods for enhancing beta cell survival and/or for stimulating beta cell proliferation, comprising co-transplanting pancreatic islets, beta cells, and/or stem cells which can generate beta cells, with (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells. The present invention is further direct to methods for reinnervation in organ or tissue transplant patients, comprising co-transplanting with the organ or tissue (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells. Kits for conducting such methods are also encompassed by the invention.

Within the context of the present disclosure, the term “co-transplantation” encompasses the simultaneous transplantation of the indicated materials, and the sequential transplantation of the indicated materials, in any order, as long as the individual transplantations are sufficiently close in sequence to provide the improved cell survival and/or proliferation, and/or improved reinnervation.

Within the context of the present disclosure, the term “stem cells” encompasses any stem cell, including, but not limited to, embryonic stem (ES) cells, adult stem cells and tissue progenitor cells, somatic cell nuclear transfer, single cell embryo biopsy, arrested embryos, altered nuclear transfer, reprogramming cells, autoimmune fluid-derived stem cells, or the like.

As noted above, the endocrine pancreas normally possesses a rich innervation consisting of both sympathetic, parasympathetic, sensory, peptidergic, and nitric oxide synthase-containing neurons which help to modulate endocrine secretion, but after implantation, however, most nerves degenerate and are only slowly replaced by nerves growing in from surrounding structures, mainly in association with blood vessels. Furthermore, it seems as if neurons present in islet grafts do not survive for more than up to a week after transplantation. As also noted above, an important contributor is the immediate post-transplantation cell death due to hypoxia, leading to a decrease in the number of engrafted beta cells. Accordingly, a first embodiment of the invention is directed to overcoming this condition, and is based on the discovery that (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells can enhance beta cell survival and/or stimulate beta cell proliferation in islet, beta cell or beta-cell producing cell (i.e., beta-cell differentiating ES cells) transplantations.

The neural crest gives rise to diverse types of cells (Le Douarin et al, “Multipotentiality of the neural crest,” Curr Opin Genet Dev, 13:529-536 (2003)). Neural crest cells generate a variety of sensory and autonomic neurons as well as glial cells of the peripheral nervous system, pericytes and smooth muscle cells of the vascular system, including the major vessels of the heart, chromaffin cells (endocrine cells of the adrenal gland), and most pigment cells. In addition, neural crest cells originating from the developing head give rise to connective tissue of the cranial muscles and chondrocytes, osteoblasts and odontoblasts, and components of the craniofacial skeleton. Neural crest cells are present in target tissues such as the ectoderm, the sympathetic ganglia, the sensory ganglia, the enteric nervous system, and the cardiac outflow tract.

Recently, signals specifying sensory neurons from neural crest cells have been described. Thus, a number of studies have demonstrated the importance of certain transcription factors in dorsal root ganglia (DRG) determination. Several basic helix-loop-helix and homeodomain proteins have been identified as proneural or neurogenic transcription factors involved in neuronal specification of the early stages of determination and lineage-specific terminal differentiation (Guillemot, “Cell fate specification in the mammalian telencephalon,” Prog Neurobiol, 83:37-52 (2007); Guillemot, “Spatial and temporal specification of neural fates by transcription factor codes,” Development, 134:3771-3780 (2007)).

In one embodiment of the methods of the invention, pancreatic islets, beta cells, and/or stem cells which can generate beta cells, are co-transplanted with neural crest stem cells (NCSCs). In a specific embodiment of the inventive methods, the NCSCs may be boundary cap NCSCs (bNCSCs). Unlike sciatic nerve neural crest stem cells, the boundary cap NCSCs generate sensory neurons upon differentiation. The bNCSCs constitute a common source of cells for functionally diverse types of neurons. Recently, the boundary cap neural crest stem cells (bNCSC) have been shown to give rise to the subtype of sensory neurons (Hjerling-Leffler et al, “The boundary cap: a source of neural crest stem cells that generate multiple sensory neuron subtypes,” Development, 132:2623-2632 (2005)), and differentiation of bNCSCs towards sensory neurons can be conditionally regulated from the outside (i.e., externally) after their transplantation to the recipient (Aldskogius et al, “Regulation of boundary cap neural crest stem cell differentiation after transplantation,” Stem Cells, 27:1592-1603 (2009)). The neural crest is therefore a suitable neural stem cell source for co-transplantation with stem cells (including ES cells) to support survival and function in cell replacement therapy. The present methods thus offer a means to guide co-transplanted neural crest stem cells to a desired type of neurons/glia. In a specific embodiment, the bNCSCs are human cells. In additional embodiments, the NCSCs are from other, non-human species, and specifically, the bNCSCs are from other, non-human species.

In a further specific embodiment of the method for treating a type 1 diabetic or type 2 diabetic patient, the method comprises co-transplanting human pancreatic islets, beta cells, and/or stem cells which can generate beta cells, with neural crest stem cells (NCSCs), more specifically bNCSCs, or, more specifically, human bNCSCs.

Tetracycline-regulated gene expression system (Tet-System)-containing neural stem/progenitor cells (TetStock neural stem/progenitor cells) are transfected stem/progenitor cells with conditionally regulated gene expression systems that can be activated from the outside, i.e., externally, after transplantation to guide the differentiation of transplanted stem/progenitor cells to the desired type of cells. These cells are described in WO 2008/002250 (PCT/SE2007/000636), incorporated herein by reference in its entirety. The TetStock neural stem/progenitor cells possess regulated activation of neural stem/progenitor cell survival/differentiation factors and according to the inventive methods may be co-transplanted with pancreatic islets, beta cells or stem cells which generate beta cells, i.e., ES cells which differentiate to beta cells. As described in WO 2008/002250, an extrinsic gene regulating system is used for enhancing survival and controlling differentiation of transplanted neural stem cells. In specific embodiments, the TetStock neural stem/progenitor cells may comprise ES cells, adult stem cells and tissue progenitor cells, somatic cell nuclear transfer, single cell embryo biopsy, arrested embryos, altered nuclear transfer, reprogramming cells, autoimmune fluid-derived stem cells, or the like. Furthermore, the TetStock neural stem/progenitor cells can be employed to guide the differentiation of any type of stem cell, including ES cells, to desired cell type(s) for which the key transcription factors are known. Specifically, the TetStock neural stem/progenitor cells can be guided to differentiate to the specific type of sensory neurons, autonomic neurons or glial cells that is desirable for optimal reinnervation and survival of the co-transplanted cells in a specific situation.

Thus, TetStock the neural stem/progenitor cells prepared for co-transplantation with pancreatic islets, beta cells or with stem cells which can generate beta cells, include ES cells, adult stem cells and tissue progenitor cells, somatic cell nuclear transfer, single cell embryo biopsy, arrested embryos, altered nuclear transfer, reprogramming cells, autoimmune fluid-derived stem cells, modified by the transfection with a gene regulating expression system which allows the sequential activation of transcription factors after transplantation. This activation of specific transcription factors can guide the differentiation of the stem/progenitor cells or the conditional expression of survival and growth-supporting genes to occur at a desired time. This approach may lead to more efficient outcome of the co-transplantation procedure. In addition, TetStock neural stem/progenitor cells (ii) may be delivered to the co-transplanted cells mentioned above, to synchronize different aspects of their differentiation, in correlation or combination with specific innervation from co-transplanted NCSCs (i), or, more specifically, boundary cap neural crest stem cells.

In a specific embodiment, the TetStock neural stem/progenitor cells differentiate to sensory neuron subtypes, autonomic neuron subtypes and/or glial cell subtypes.

In another specific embodiment, the beta-cell generating cells, i.e., ES cells differentiating to beta cells, can also be provided as transfected stem/progenitor cells having a conditional externally-regulated gene expression system, i.e., as TetStock stem/progenitor cells. Thus, in a more specific embodiment, the method comprises co-transplantation of TetStock neural stem/progenitor cells with TetStock beta-cell differentiating stem cells.

In yet another embodiment, pre-differentiated stem/progenitor neural cells may be employed. The cells may be sensory neuron subtypes, autonomic neuron subtypes, and/or glial cell subtypes. Various methods known in the art may be employed to generate pre-differentiated stem/progenitor neural cells, including the Tet-systems known in the art, for example, as described in the aforementioned WO 2008/002250.

The present inventors have discovered that co-transplantation as described herein enhances beta cell proliferation and function, after transplantation and in vivo. In a specific embodiment of the methods of the invention, boundary cap neural crest stem cells are co-transplanted with pancreatic islets. The present methods are useful for improving long term viability of differentiated beta cells as well as for cells which are derived from ES cells, or any other type of stem cells, generating beta cells. These methods are particularly advantageous for use in treating patients with type 1 diabetes or insulin-deficient type 2 diabetes.

According to another aspect of the invention, the above-mentioned methods of enhancing beta cell survival and proliferation during and after implantation may also be used as a therapeutic method for treating patients with diabetes. The treatment may be achieved by transplanting pancreatic islets or any type of stem cells which produce beta cells, either before or after transplantation, to the patients together with NCSCs. These therapeutic methods produce neurotrophic support and specific innervation of pancreatic islets and/or newly differentiated beta cells.

According to a further aspect of the invention, the therapeutic method may be directed to patients requiring organs and tissues to be reinnervated after transplantation, for example in conjunction with myocardial transplantation, liver transplantation, or other organ transplantation, or newly created organs/tissues derived from stem/progenitor cells of different sources, including, but not limited to, somatic cell nuclear transfer, single cell embryo biopsy, arrested embryos, altered nuclear transfer and reprogramming somatic cells. These methods comprise using, in addition to stem cells, one or more of the following cell types: co-transplantation with the organ or tissue of bNCSCs, TetStock neural stem/progenitor cells, and/or in vitro pre-differentiated stem/progenitor neural cells, for example, sensory neuron subtypes, autonomic neuron subtypes, and/or glial cell subtypes.

According to yet another aspect, the present invention relates to kits for use in the described methods. According to one specific embodiment, the kit is devised for co-transplantation with pancreatic islets, beta cells, or stem cells which generate, i.e., differentiate to, beta cells, and may as such comprise one or more of the following components: (i) neural crest stem cells (NCSCs), (ii) tetracycline-regulated gene expression system (Tet-System)-containing neural stem/progenitor cells (TetStock neural stem/progenitor cells), including for sensory neuron subtypes, autonomic neuron subtypes, and/or glial cell subtypes, and/or (iii) pre-differentiated stem/progenitor neural cells, including for sensory neuron subtypes, autonomic neuron subtypes, and/or glial cell subtypes.

According to another embodiment, the kit is devised for a method of reinnervation of organs after transplantation or organs/tissues created from stem/progenitor cells of different sources. The kit comprises, in addition to stem cells (including ES cells), one or more of the following cell types: (i) neural crest stem cells (NCSCs), (ii) tetracycline-regulated gene expression system (Tet-System)-containing neural stem/progenitor cells (TetStock neural stem/progenitor cells), including for sensory neuron subtypes, autonomic neuron subtypes, and/or glial cell subtypes, and/or (iii) pre-differentiated stem/progenitor neural cells, including for sensory neuron subtypes, autonomic neuron subtypes, and/or glial cell subtypes. In one specific embodiment of the inventive kits, the NCSCs may be boundary cap NCSCs (bNCSCs). In another specific embodiment, the bNCSCs are human cells. In additional embodiments of the kits, the NCSCs are from other, non-human species, and specifically, the bNCSCs are from other, non-human species.

In one embodiment, the described methods and kits comprise human cells, while in alternative embodiments may comprise cells derived from animals, and such methods and kits may be used for the corresponding veterinary purposes.

In the experiments described in the Example herein, beta cell survival and proliferation as well as islet function were analyzed in in vitro and in vivo experimental models. The Example demonstrates that islets extensively proliferate in vitro in the presence of NCSCs. Particularly, the proliferation of beta cells was extensive after 3 days in co-culture. Results from a trans well in vitro assay suggest that soluble factors are responsible for the enhanced proliferation of beta cells in vitro. Importantly, these factors originate not from the NCSCs when they are alone, but when NCSCs are in direct contact with the islet cells. The soluble factors produced by these co-cultures induced proliferation of beta cells in the compartment of the trans well assay that was separated from co-cultured islets and NCSCs.

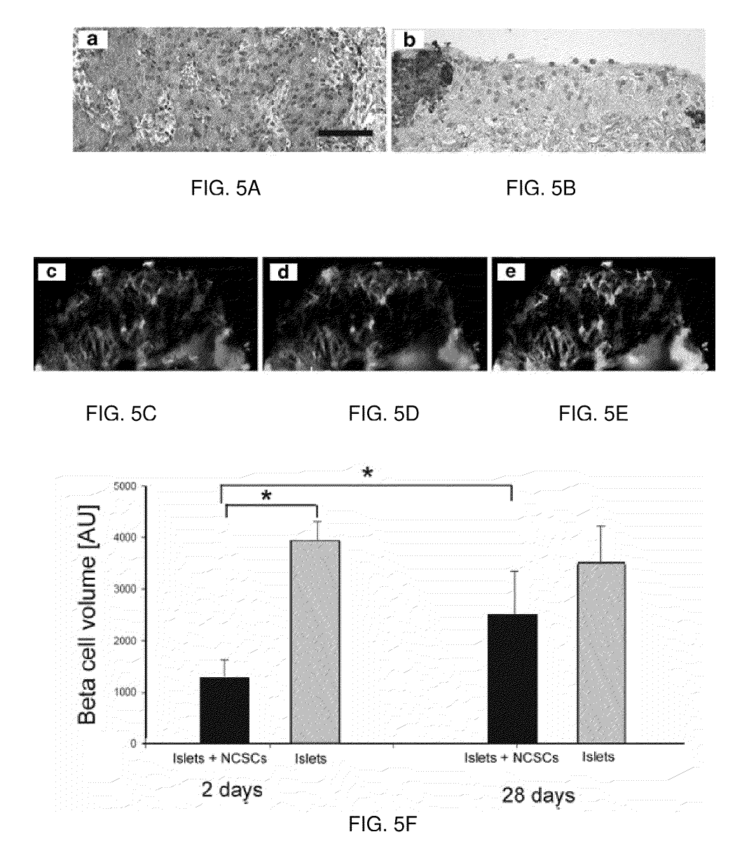

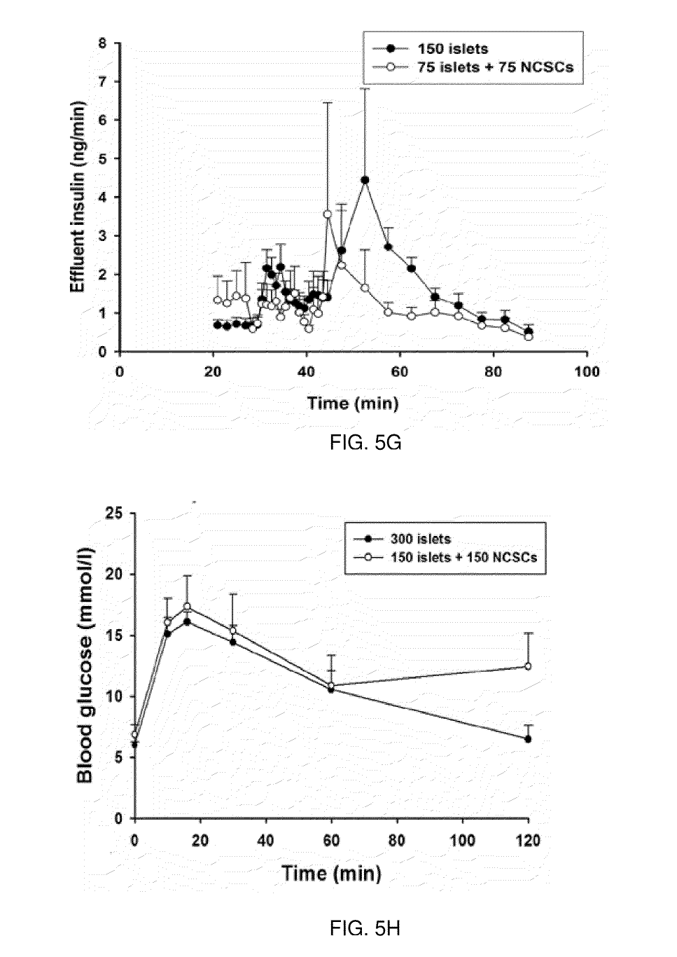

The in vivo experiments demonstrate that islet cells extensively proliferate during the first week after co-transplantation with the NCSCs; particularly, beta cells proliferated extensively in these mixed transplants (see FIG. 4H and the related discussion in the Example). Thus, the enhanced long term survival of beta cells in mixed transplants was due to their extensive proliferation at earlier stages. A faster insulin release from mixed transplants was also observed, which may reflect an earlier functional maturation of secretory granules in the beta cells of mixed transplants (see FIG. 5B and the related discussion in the Example).

The implanted bNCSCs remain viable after implantation to normoglycaemic or alloxan-diabetic mice, both when implanted alone and together with islets. However, the transplant volume in bNCSC single grafts is strikingly small with only few cells at the initial graft site. This is not due to poor survival of grafted bNCSCs, however, but rather to their extensive migration towards the islet transplants in the other pole of the kidney. Interestingly, bNCSCs did not migrate out from mixed transplants, but remained in the vicinity of neighboring beta cells, strongly indicating that islets exerted an attractive influence on bNCSCs. In vitro co-culture experiments with fluorescent beta cells from DsRed mice and bNCSCs from eGFP mice confirmed that already in 6 hours, NCSCs had migrated towards islet cells and extensively surrounded them.

Some of the bNCSCs were positive for the neuronal marker bTUB, but most of the transplanted cells expressed the glial marker GFAP. Of interest in this context is that endogenous pancreatic islets are ensheathed by Schwann cells (Donev, “Ultrastructural evidence for the presence of a glial sheath investing the islets of Langerhans in the pancreas of mammals,” Cell Tissue Res, 237:343-348 (1984)), and factors from the islets may help to stimulate the differentiation of bNCSCs in this direction. Experiments have confirmed that indeed the bNCSCs differentiate towards neurons in the presence of islets in vitro and in vivo (Kroon et al (2008)). However, transplanted bNCSCs and islets were always clearly separated from each other, suggesting that their interactions occurred through diffusible factors rather than by cell-cell contact.

An alternative to islet transplantation as described in the Example to achieve long term survival of functional grafts is the use of undifferentiated stem/progenitor cells, which are able to differentiate to beta cells in the recipient. A recent study has demonstrated successful differentiation of human embryonic stem cells to functional beta cells after transplantation to immune-compromised mice (Kroon et al (2008)). The Example herein shows that the boundary cap neural crest stem cells exert a potent stimulatory effect on growth and function of beta cells in vitro as well as in co-grafts with pancreatic islets in vivo. The present methods similarly include co-operation between co-transplanted neural crest stem cells and other stem cells, including ES cells, of potential use for cell replacement therapy and/or organ or tissue repair.

Beta cells derived from ES cells or other stem cells will be without extrinsic as well as intrinsic innervation for a considerable period of time. Co-transplanted neural crest stem cells may provide initial trophic support to beta cells and subsequent innervation for their long term maintenance and adequate function.

### EXAMPLE

### Co-Transplantation of bNCSCs and Pancreatic Islets in Mice

This Example demonstrates the improvement in cell survival and proliferation resulting from co-transplantation of pancreatic islets and bNCSCs.

Animals

Transgenic heterozygous C57BL/6-b-actin-enhanced green fluorescent protein (eGFP) mice (Jackson Laboratories, Bar Harbor, Me., USA) were used for isolation of neural crest stem cells (NCSCs). Pancreatic islets were isolated from C57BL/6 mice (B&M, Ry, Denmark). Male C57/BL/6 nu/nu mice (B&K) were used as graft recipients. All procedures were approved by the Regional Ethical Committee for Research on Animals.

Preparation of Neurospheres

Dorsal root ganglia (DRGs) from 11.5 day old eGFP mouse embryos were isolated, and used for setting up neural crest stem cell (NCSC) cultures from the so-called boundary cap (bNCSCs) (Hjerling-Leffler J, et al. (2005)). Briefly, the uterus was removed from the anaesthetized pregnant mouse and placed in cold phosphate-buffered saline (PBS). Embryos were separated, rinsed in PBS, placed in N2 medium and the DRGs were removed and collected in N2 medium. Collected DRGs were allowed to settle down before removing the supernatant and adding a Collagenase/Dispase (1 mg/ml) and DNase (0.5 mg/ml) solution in N2 and incubating for 20-30 minutes in a 37° C. water bath, followed by rinsing in N2 medium with B27 (1:50) and plating ˜1-2×105 cells/well in a 24-well dish after dissociation. Cells were placed directly into 500 μl N2 medium containing B27, epidermal growth factor (EGF; 20 ng/ml), and basic fibroblast growth factor (bFGF; 20 ng/ml). After 12 h, non-adherent cells were removed together with half of the medium before adding up to 250 μl of fresh medium. The medium was then changed every other day (50% of the medium replaced with fresh medium) before neurospheres began to form. For the experiments, neurospheres from passage 4-5 were collected as described in next paragraph.

Islet Isolation

Pancreatic islets were isolated from C57BL/6 mice by a collagenase digestion method, as previously described (Le Douarin (2003)). The islets were cultured free-floating for three-five days, with 150 islets in each culture dish, in 5 ml culture medium, RPMI 1640 (Sigma-Aldrich, Irvine, UK) supplemented with L-glutamine (Sigma-Aldrich), benzylpenicillin (100 U/ml; Roche Diagnostics Scandinavia, Bromma, Sweden), streptomycin (0.1 mg/ml; Sigma-Aldrich) and 10% (v/v) fetal calf serum (Sigma-Aldrich). The medium was changed every second day.

Co-Culture Experiments

Islets alone (n=10) or together with NCSCs (neurospheres) in equal proportions (n=5 of each), corresponding to approximately 3×104 cells/well were cultured in propagation or differentiation medium on 50 μg/ml poly-D-Lysine and 20 mg/ml laminin (Sigma-Aldrich) coated coverslips for seven days. Cells were photographed daily.

In Vitro Insulin Secretion Assay

Glucose-stimulated insulin secretion in 1 week cultures of islet-alone and NCSC-islet co-cultures was assessed. Medium was removed and cells were placed for 60 min at 37° C. with culture medium containing 2 mM glucose, changed to a buffer supplemented with 1 mg/ml BSA (fraction V; Boehringer Mannheim GmbH) for 30 min at 37° C., changed again to the same type of buffer containing either 2 or 20 mM glucose. After incubation at 37° C. for 30 min the aliquots of the buffer were taken for determination of released insulin and the cells were subsequently washed and lysed in insulin release buffer to determine insulin content. Released insulin and insulin content were determined after appropriate dilutions with ELISA, as described previously (Bergsten et al, “Glucose-induced amplitude regulation of pulsatile insulin secretion from individual pancreatic islets,” Diabetes, 42:670-674 (1993)). Experiments were repeated 4 times (8 wells in each experiment).

Transwell Assay

To investigate if proliferation of islet cells was induced by direct contact with the NCSCs, or through soluble factors produced by NCSCs, we performed transwell assay where islets were placed on the bottom and NCSCs alone or a mixed culture of NCSCs and islets were placed on the top of the cell impermeable membrane (Falcon, Cat. Number 353104). Experiments were performed with or without mitogens in the culture medium. In some cases, the membrane of trans well chamber was coated with poly-D-lysine and laminin similar to the cover slips. The cultures were photographed daily, and after seven days cover slips were removed and proliferation of islet cells was assessed.

Transplantation Procedures

Male C57/BL/6 nu/nu mice were anaesthetized with an intraperitoneal injection of avertin. The kidneys were exposed through a flank incision and a total of 3 grafts were implanted in each animal. In the right kidney 150 islets (upper pole) and 150 neurospheres (lower pole) were implanted whereas in the left kidney 75 islets+75 neurospheres were implanted mixed together into one graft. The animals were allowed to recover.

Perfusion of Grafts

Animals used for these studies were anesthetized with avertin one month after transplantation. All 3 grafts were identified and a small incision was made in the renal capsule immediately adjacent to each of the grafts. By carefully lifting the capsule, this could be removed together with the grafts. The transplants were then perfused to assess their insulin secretion in response to glucose stimulation (Tyrberg et al, “Species differences in susceptibility of transplanted and cultured pancreatic islets to the beta-cell toxin alloxan,” Gen Comp Endocrinol, 122, 238-251 (2001)). Briefly, islet grafts consisting of 150 islets or 75 islet+neurosphere grafts (75 of each) or 150 neurospheres were placed in small chambers (˜1 mm³) with a bottom consisting of a polyamide net (mesh size 25 μm) and perfused with 1 ml/min of KRBH with the addition of 1% bovine serum albumin (BSA) and 2.8 mmol/l D-glucose for 30 min. After this normalization period a solution of KRBH+1% BSA with 28 mmol/l D-glucose was perfused for 30 min followed by KRBH+BSA with 2.8 mmol/l D-glucose for 20 min. Samples were taken for analysis of insulin every minute from 20 min and onwards. Insulin was analyzed with ELISA (Mouse Insulin ELISA; Mercodia AB, Uppsala, Sweden).

Immunohistochemistry

After the graft perfusions, transplants were fixed for two hours in 4% formaldehyde (v/v) and 14% saturated picric acid (w/v) in phosphate-buffered saline (PBS) (ca. 4° C.; pH 7.4), left over night in PBS containing 15% sucrose and cut on cryostat in 12 μm thick sections. These were pre-incubated with blocking solution (1% BSA, 0.3% Triton X-100 and 0.1% NaN³ in PBS) for one hour at room temperature and then incubated overnight at 4° C. with primary antibodies for insulin (guinea pig polyclonal, 1:250, DAKO) to label beta cells, β-tubulin class III (bTUB, mouse monoclonal, 1:500, Covance) to identify transplanted NCSCs, which had differentiated to neurons, anti-calcitonin gene-related peptide (CGRP; rabbit polyclonal, 1:4000, Chemicon) and antibody RT97 (mouse monoclonal, 1:500, Immunkemi) for sensory neuron subtypes, and anti-glial fibrillary acidic protein (GFAP; rabbit polyclonal, 1:400, DAKO) for glial cells. After washing with PBS, appropriate secondary antibodies (Jackson ImmunoResearch, UK) were applied for four hours at room temperature: Cy3 conjugated donkey anti-mouse and anti-guinea pig (1:500), AMCA-conjugated donkey anti-rabbit (1:100). Sections were rinsed three times in PBS for 15 minutes, with the second wash in some sections including Hoechst 33342; 11 ng/ml, Molecular Probes, and mounted in a mixture of PBS and glycerol (1:1; v/v) containing 0.1M propyl-gallate.

Morphological Evaluation

For evaluation of transplant size and beta cell survival, every 5th section was photographed (n=4 each group). The NIH software ImageJ (available at http://rsb.info.nih.gov/ij) was used to measure transplant areas. Estimates of the entire transplant volume as well as the volume of the beta cell compartment in the transplant were calculated according to the formula A=TK[Σ(S1 to Sn)], where T is the thickness of the section (T=12 μm), K is the number of sections between the measured areas (K=5) and S is the area of the transplant on the sections from 1 to N.

Statistical Calculations

Values given are means±SEM. Probabilities (P) of chance differences were calculated with Student's unpaired t-test or Wilcoxon's rank-sum-test. P-values <0.05 were considered to be statistically significant.

Survival and Differentiation of bNCSCs Transplanted to the Kidney

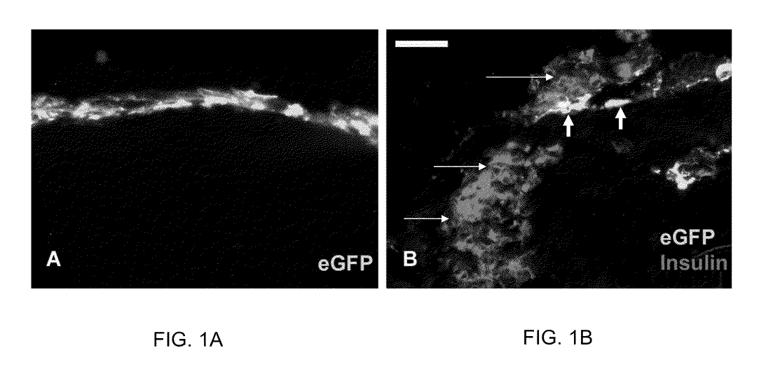

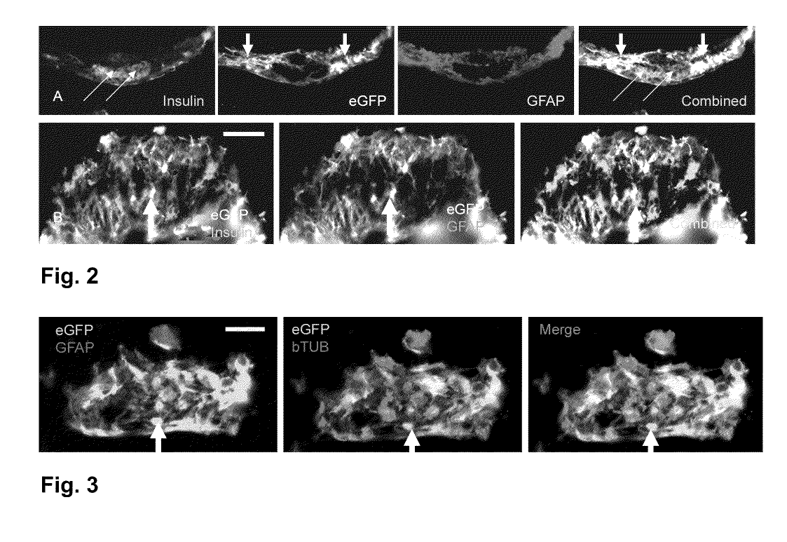

One month after transplantation, eGFP-expressing bNCSCs were observed at the lower pole of the left kidney as well as under the capsule towards the islet transplant at the upper pole of the same kidney (FIG. 1A). Some of the migrated bNCSCs reached the vicinity of the islet cells, but were located separately and did not surround the islet cells (FIG. 1B). In the other kidney, containing mixed islet and eGFP-expressing bNCSC grafts, the bNCSCs often surrounded groups of islet cells, but in the majority of transplants, distinct boundaries between the two cell types were still maintained (FIG. 2; FIGS. 4A, 4B). No migrating bNCSCs were observed, either in kidney capsule or in the kidney parenchyma when bNCSCs were implanted together with the islet transplants in the same kidney.

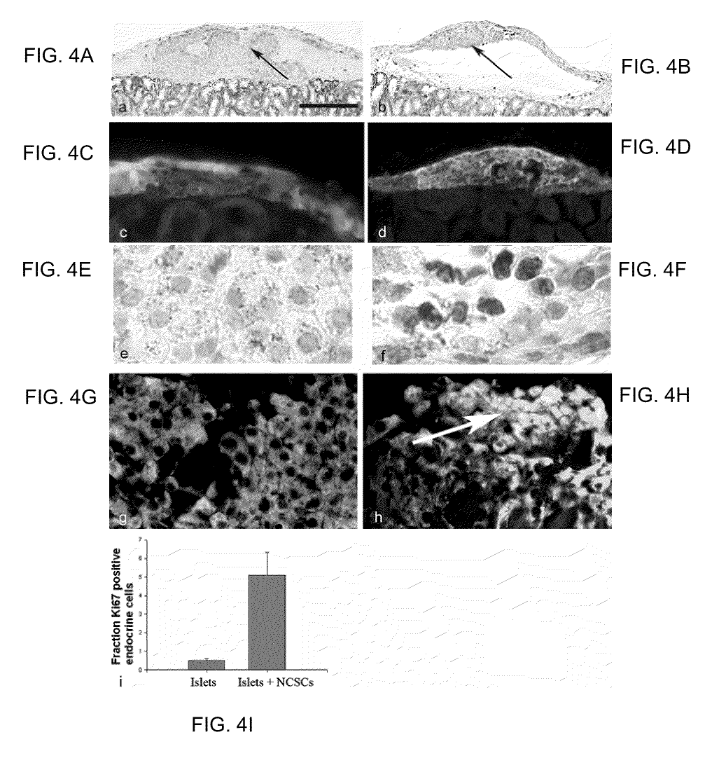

Double labeling of eGFP expressing bNCSCs with the neuronal marker bTUB and glial marker GFAP revealed a predominant differentiation of transplanted stem cells towards glial type (FIG. 2). However some bTUB expressing cells were also present, but not in association with the islets (FIG. 3). No double labeling was observed between bTUB and RT97, a marker for low threshold mechanosensory neurons or anti-CGRP, a marker for peptidergic nociceptive neurons, indicating that transplanted bNCSCs did not differentiate to DRG neuron phenotypes (not shown).

Survival and Function of Beta Cells Transplanted to the Kidney

The transplants were easily identified in the kidneys of the recipient animals both at two days and at one month after transplantation. Islet-alone grafts were identified at the place of the grafting whereas in the kidney containing a mixture of islets and NCSCs, the grafts often occupied a larger area under the kidney capsule and looked flatter than islet-alone transplants. The morphology of the islet grafts two days after transplantation demonstrated a compact mass of endocrine cells (FIG. 4A), sometimes with a central core of loose connective tissue whereas mixed grafts often contained a single fluid-filled cyst in the central portion of the grafts (FIG. 4B).

The interrelations between beta cells and NCSCs in mixed grafts developed early after transplantation. After two days, the NCSCs were located at the periphery of the tightly packed insulin-positive areas and were extensively dispersed in different directions from the site of transplantation under the kidney capsule. In some cases, the NCSCs attached to insulin-positive cells (FIG. 4C), or were in close proximity to islet cells (FIG. 4D). At this stage the NCSCs extensively expressed GFAP and showed almost complete overlap with EGFP natural staining (not shown). GFAP-positive extensions from the NCSCs to some parts of the insulin-positive areas (FIG. 4H) were registered. In the case of cyst formation, the NCSCs covered the entire inner surface of the cysts, thus isolating the islet cells from the fluid.

TUNEL or Ki67 staining was made on adjacent insulin labeled sections. No difference in the incidence of TUNEL positive cells was seen in islet-alone compared to mixed grafts (0.19±0.03 vs. 0.21±0.04% of the endocrine cells, respectively) (not shown). In contrast, the number of Ki67 positive endocrine cells within mixed grafts was strongly increased (FIGS. 4E and 4F) and was 10 times higher compared to islet-alone transplants (FIG. 4I). To determine whether β-cells contributed to the proliferation in the endocrine areas of mixed transplants, triple immunofluorescence labeling with antibodies to insulin, the proliferation marker Ki67 and GFAP was performed.

These results showed that insulin-positive cells extensively contributed to the population of proliferating cells in the endocrine areas of mixed transplants, whereas only occasional dividing insulin-negative cells were found in islet-alone transplants (FIGS. 4G and 4H). The insulin-positive proliferating cells in mixed transplants were located in clusters and cells were smaller than insulin-positive cells in neighboring areas or in islet-alone transplants (FIG. 4H). At this stage the insulin-positive cell volume in mixed grafts was smaller than that of islet-alone grafts in line with the smaller number of transplanted islets in the mixed grafts (FIG. 4F).

One month after transplantation, large areas in islet-alone as well as in mixed transplants were occupied by insulin-positive or by insulin-negative endocrine cells (FIGS. 5A and 5B, respectively). The NCSCs were found in direct vicinity to the insulin-positive cells (FIG. 5C-5E) and extensively expressed the glial marker GFAP or the neuronal marker bTUB. The proportion of insulin-positive cells of the entire graft size constituted 31±6% of islet-alone (n=6) and 12±3% of mixed (n=7) grafts (P<0.027 when calculated with Wilcoxon's rank-sum test). However, the beta cell volume within the grafts was similar in islet-alone and in mixed grafts (FIG. 5F; P=0.42). The beta cell volume in mixed one-month transplants was significantly increased compared to the beta cell volume in two-days transplants (FIG. 5F; P=0.037).

Stimulation of the grafts with a high glucose concentration in perifusion experiments induced a biphasic release of insulin from both islet-alone and mixed grafts with no differences in the total amount of insulin released from the grafts as evidenced by similar values for area under the curve when insulin release was plotted against time (FIG. 5G). The initiation of insulin release was, however, significantly faster in mixed transplants (FIG. 5G).

Alloxan-diabetic mice treated with a graft of 300 islets showed a normoglycaemic response in the glucose tolerance test one month after grafting. Alloxan-induced recipients receiving mixed grafts (150 islets and 150 neurospheres) showed similar response, although blood glucose values tended to be higher at 120 minutes, but the difference was not significant (FIG. 5H, P=0.074).

In 7 alloxan-diabetic graft recipients that had been cured by an islet-alone or mixed graft, the transplants (n=3 and n=4 respectively) were removed and diabetes developed as confirmed with blood tests showing glucose levels above 11.1 mmol/after nephrectomy.

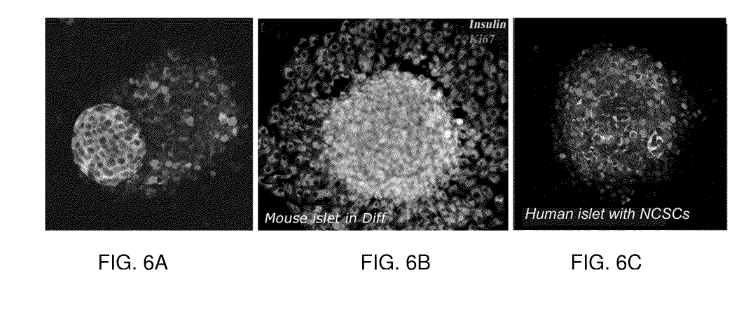

In vitro experiments have demonstrated that in co-cultures, only half of the amount of islets (n=5) produce the same amount of insulin as islets in the islet-alone cultures (n=10) (insulin-secretion assay, not shown). Most important was the finding that proliferation of beta cells was strongly enhanced in the presence of the NCSCs (FIGS. 6A-6C). In trans well assay, the proliferation of beta cells in islets-alone compartment was only found when this compartment was separated by permeable membrane with the NCSCs-islet co-cultures. This finding suggests that the stimulation of the proliferation of beta cell factor is a soluble factor, which is produced by collaboration of islet with the NCSCs.

Additional pilot experiments with the human islets showed that the co-cultures with the mouse NCSCs stimulates the proliferation of human islet cells with some of them being the beta cells (FIG. 6C).

The specific descriptions, examples and embodiments described herein are exemplary only in nature and are not intended to be limiting of the invention defined by the claims. Further embodiments and examples, and advantages thereof, will be apparent to one of ordinary skill in the art in view of this specification and are within the scope of the claimed invention.

What is claimed is:

1. A method for enhancing beta cell survival and/or for stimulating beta cell proliferation, comprising co-transplanting pancreatic islets, beta cells, and/or stem cells which can generate beta cells, with (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells.

2. The method of claim 1, comprising co-transplanting human pancreatic islets, beta cells, and/or stem cells which can generate beta cells, with human neural crest stem cells (NCSCs).

3. The method of claim 1, comprising co-transplanting human pancreatic islets, beta cells, and/or stem cells which can generate beta cells, with human boundary cap neural crest stem cells (bNCSCs).

4. The method of claim 1, wherein pancreatic islets are transplanted.

5. The method of claim 1, wherein beta cells are transplanted.

6. The method of claim 1, wherein stem cells which can generate beta cells are transplanted.

7. The method of claim 1, comprising co-transplanting human pancreatic islets, beta cells, and/or stem cells which can generate beta cells, with TetStock neural stem/progenitor cells for sensory neuron subtypes, autonomic neuron subtypes and/or glial cell subtypes.

8. The method of claim 1, comprising co-transplanting human pancreatic islets, beta cells, and/or stem cells which can generate beta cells, with pre-differentiated stem/progenitor neural cells for sensory neuron subtypes, autonomic neuron subtypes and/or glial cell subtypes.

9. The method of claim 1, wherein the co-transplantation is to a human patient diagnosed with diabetes.

10. The method of claim 9, wherein the diabetes is type 1 diabetes.

11. The method of claim 9, wherein the diabetes is insulin-deficient type 2 diabetes.

12. A method for treating a type 1 diabetic or type 2 insulin-deficient diabetic patient, comprising co-transplanting human pancreatic islets, beta cells, and/or stem cells which can generate beta cells, with neural crest stem cells (NCSCs).

13. The method of claim 12, wherein the NCSCs are boundary cap NCSCs (bNCSCs).

14. The method of claim 12, wherein the bNCSCs are human bNCSCs.

15. A kit for enhancing beta cell survival and/or for stimulating beta cell proliferation, comprising pancreatic islets, beta cells, and/or stem cells which can generate beta cells, and (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells.

16. A method for reinnervation in an organ or tissue transplant patient, comprising co-transplanting with the organ or tissue (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells.

17. The method of claim 16, wherein the organ or tissue comprises stem cells.

18. The method of claim 16, wherein the patient is an organ transplant patient.

19. The method of claim 16, wherein the patient is a tissue transplant patient.

20. A kit for reinnervation in a stem-cell containing organ or tissue transplant patient, comprising a stem-cell containing organ or tissue, and (i) neural crest stem cells (NCSCs), (ii) tetracycline-regulated gene expression system (Tet-System)-containing neural stem/progenitor cells (TetStock neural stem/progenitor cells), and/or (iii) pre-differentiated stem/progenitor neural cells.
